(19) United States
(12) Patent Application Publication
(10) Pub. No.: US 2008/0228026 A1
(43) Pub. Date: Sep. 18, 2008

(54) Method and apparatus for image enhanced external counterpulsation

(75) Inventors: Loren A. Manera, Laguna Hills, CA (US); Kenneth L. Burns, Laguna Hills, CA (US); William R. Malenius, Laguna Hills, CA (US)

Correspondence Address: Crockett & Crockett, Suite 400, 24012 Calle De La Plata, Laguna Hills, CA, 92653, US

(73) Assignee: Applied Cardiac Systems, Inc.

(21) Appl. No.: 12/075,869

(22) Filed: Mar. 14, 2008

Related U.S. Application Data
(60) Provisional application No. 60/918,113, filed on Mar. 14, 2007.

(51) Int. Cl. A61M 1/10; A61B 8/00

(52) U.S. Cl. 600/17; 600/439

(57) ABSTRACT

An image enhanced external counterpulsation system may be used to monitor a continuous real-time ultrasound image of the patient's heart during ECP therapy.

### RELATED APPLICATIONS

This application claims priority from copending U.S. Provisional patent application 60/918,113 filed Mar. 14, 2007.

### FIELD OF THE INVENTIONS

The inventions described below relate to the field of medicine and more specifically to medical diagnosis and therapy using simultaneous ultrasound, ECG, tonometry and external counterpulsation.

### BACKGROUND OF THE INVENTIONS

Echocardiography has been used for noninvasive imaging of a heart in operation. Use of this technique has generally been limited to office or laboratory environments with the patient generally immobile (laying, sitting or standing).

Conventional External Counter Pulsation (ECP) treatment usually lasts 35 days with the results of the therapy being subjective, usually based on how the patient describes their current feeling of well being and overall discomfort relative to how they felt prior to the beginning treatment. Furthermore, the only functions monitored continuously during conventional ECP treatment are ECG and photo-electro plethysmograph waveforms. Blood pressure and pulse oxide are measured before and after individual therapy sessions. Currently, ultrasound is used only prior to the 35 days of treatment for patient screening and safety. Usually to detect contraindications such as aortic insufficiency, abdominal aneurisms, mitral valve regurgitation, low ejection fraction, etc. After ECP therapy ultrasound is used occasionally to measure ejection fraction and to examine for change in wall motion.

### SUMMARY

The image enhanced external counterpulsation system described below may be used to monitor a continuous real-time ultrasound image of the patient's heart during ECP therapy. The effects of preload, wall motion, tricuspid, pulmonary, mitral, and aortic valve dysfunction may be monitored as well as left ventricular dysfunction and the identification of hibernating ischemia. This system entails the observation of ejection fraction, wall motion, strain, and strain rate during therapy. Using this method and system the benefits of ECP may be directly observed during therapy and thus the duration of the therapy and parameters such as treatment pressures and inflation/deflation timing may be optimized. This technique may be used to specify additional treatment time (days) if it becomes evident that the patient has responded positively to the therapy as evidenced by the improvement in wall motion. Conversely, treatment may be terminated early if no improvement is seen.

As used herein ECP therapy, external counterpulsation therapy, may be applied as monitored by ultrasound and applanation tonometry to a patient for therapeutic benefit or for diagnostic purposes or for any suitable combination. Two or more pneumatic compression cuffs are applied to each of the patient's legs and possibly the buttocks. The ECP mechanism is synchronized with the patient's electrocardiogram such that for example, with each cardiac cycle pressure is sequentially applied distally to proximally in early diastole, resulting in an increase in diastolic blood pressure (diastolic augmentation) and retrograde aortic diastolic blood flow. At the end of diastole, pressure is released simultaneously from all cuffs, resulting in systolic unloading and afterload reduction. The ECP driver does not include vacuum assist on deflation to minimize patient movement and improve ultrasound imaging. The buttocks cuffs can be disabled individually to minimize patient movement for extreme problem cases.

Regional wall motion abnormalities of the contractile function of cardiac wall segments may be objectively determined, monitored and recorded as a baseline. Simultaneous ECP therapy, ECG and ultrasound with tonometry may be used as discussed below to objectively document a patient's condition, the immediate effects of ECP therapy and data recorded over time may permit objective determination of improvements in cardiac wall abnormalities and other conditions.

The specialized table and cushion minimizes patient motion during ECP and permits rolling of patient without compromising ECP or ultrasound. Recesses in the cushion accommodate the ECP hoses permitting the patient to be turned on their side for Ultrasound examination during ECP therapy and the hoses will not collapse or cause patient discomfort. The cushion is made of thicker foam to minimize patient bounce to facilitate good ultrasound images. In addition, the cushion may also include one or more cutouts to facilitate Ultrasound probe access to patients in the supine position for optimal probe positioning. The construction of the table may accommodate both supine, reverse Trendelenburg, and sitting positions during ECP and the Trendelenburg position if ECP is not used concurrently. The ECP hoses may recede into the pad to permit the table to be used as an Ultrasound examining table only, independent of ECP. The edge sections of the cushion are beveled up on the outside edges to keep the patient stable so Ultrasound images are clear.

An Image Enhanced External Counterpulsation system may have other practical benefits to a doctor, by combining ultrasound, ECG and tonometry with ECP in one system, the system optimizes space in a treatment room. The multifunction system may be used to perform the individual tasks of ECP therapy, ECG and or ultrasound and the table is suitable for simple patient examination.

### BRIEF DESCRIPTION OF THE DRAWINGS

FIG. 1 is a perspective view of an image Enhanced External Counterpulsation system.

FIG. 2 is a baseline ultrasound image of a heart.

FIG. 3 is an ultrasound image of the heart of FIG. 2 illustrating blood flow into the ventricle.

FIG. 4 is an ultrasound image of the heart of FIG. 2 illustrating blood flow out of the ventricle.

FIG. 5 is an ultrasound image of the heart of FIG. 2 illustrating regurgitated blood flow.

FIG. 6 is an integrated report of ultrasound data recorded during ECP therapy.

FIG. 7 is a 4-chamber tissue harmonic ultrasound image of the heart of FIG. 2.

FIG. 8 is a long axis tissue harmonic ultrasound image of the heart of FIG. 2.

### DETAILED DESCRIPTION OF THE INVENTIONS

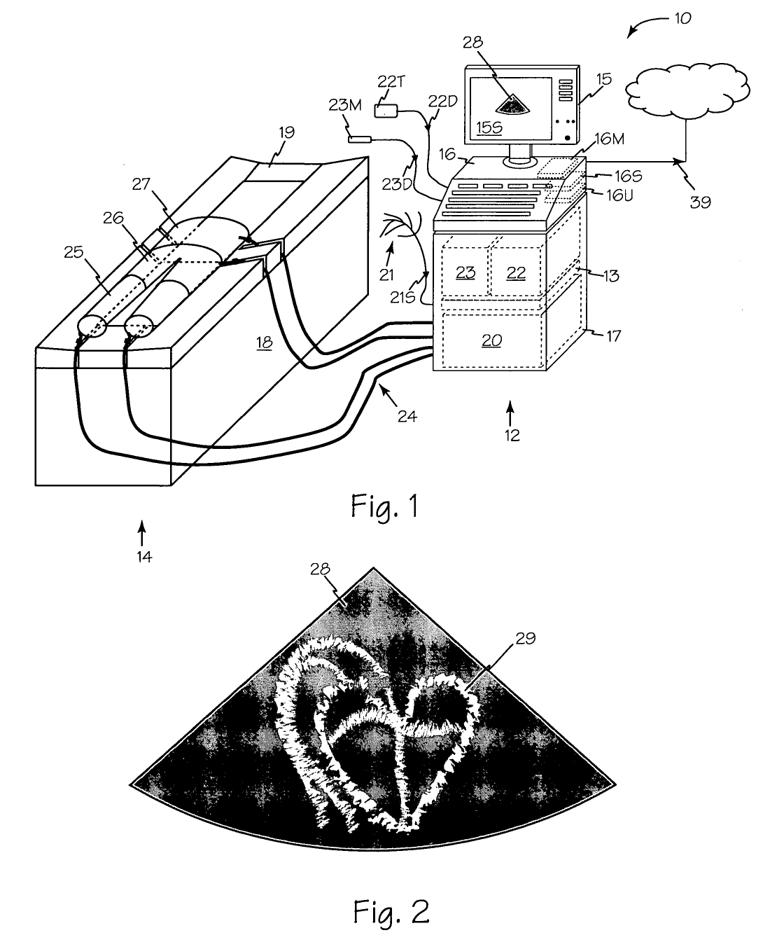

Image Enhanced External Counterpulsation system 10 of FIG. 1 includes control station 12 and table 14. Control station 12 includes monitor 15, console 16 and enclosure 17. Table 14 includes table base 18 and tabletop or cushion 19.

Console 16 controls the operation of electrocardiograph (ECG) module 13, ECP driver module 20 and ultrasound driver module 22 and also records data from the ECP driver 20, ECG module 13 and ultrasound system 22 and any other systems or modules that may be combined. ECP driver 20 provides pneumatic pressure through hoses 24 to one or more inflation cuffs such as cuffs 25, 26 and 27. Console 16 may include one or more microprocessors such as microprocessors 16U for processing ultrasound, ECP and any other suitable data.

Electrocardiograph electrodes 21 are common to both the ECP and ultrasound to ECP timing and ultrasound images are synchronized. One set of electrodes such as ECG electrodes 21 may be used, and electrical signal 21S may be divided as necessary, such as for example to separate amplifiers in ECP module 20 and ultrasound module 22.

High fidelity micromanometer 23 M and tonometry module 23 may be integrated with ECP driver 20 and ultrasound module 22 to enable highly accurate timing of the ECP therapy and provide feedback during ECP therapy to monitor marginal patients and enable ECP therapy to be applied to patients for whom it would otherwise be contraindicated. Micromanometer 23 M measures one or more circulatory system parameters and tonometry module 23 collects the circulatory system data and under control of console 16 and microprocessors 16U processes the data to provide pressure parameters that may be used to control ECP therapy. Integrated aortic blood pressure waveform analysis as performed by system software 16S, micromanometer 23 M and tonometry module 23 can be used to accurately monitor true systolic and diastolic pressures at the heart, replacing the typical photo-electro plethysmograph and may be obtained at any suitable pulse point such as for example, the wrist or the carotid artery. It can also be used to monitor efficacy of the ECP treatment at the end of some specified time frame or at the end of regular treatment period such as 35 days.

ECP, Ultrasound, and Applanation Tonometry applications may run on the same microprocessor in separate processes. Each application is designed to run multiple threads and may be optimized for dual and quad core microprocessors. Tonometry technology may provide system 10 with the following parameters:
Evaluation of systemic arterial stiffness (Aortic Augmentation Index)
Augmentation pressure; drives left ventricular load (afterload)
Central Pulse pressure in the ascending aorta (driving cerebral blood flow) giving the true pressure
Central systolic pressure; often markedly different from the cuff value.
Ejection duration—a parameter for distinguishing primarily systolic from primarily diastolic dysfunction, and for managing the efficacy ECP valve timing during therapy but should show no change when the therapy is not active.
Diastolic pressure-time index (DPTI), a key determinant of coronary blood supply.
Systolic pressure-time index (SPTI), a measure of myocardial oxygen demand.
Subendocardial Viability Ratio—the relationship of DPTI and SPTI

Integrated Ultrasound as employed in image enhanced external counter pulsation system 10 greatly enhances patient safety my allowing monitoring of borderline contraindications such as for example valvular dysfunction (mitral or aortic stenosis), aortic insufficiency, abdominal aortic aneurysm, or deep vein thrombosis. Patients can not only be monitored pre and post ECP treatment but during treatment to identify potentially dangerous contraindications and precautions that may materialize during diastolic augmentation. The following are contraindications and precautions for ECP therapy that may be waived by the use of integrated ultrasound and tonometry during ECP therapy:

### Contraindications

Arrhythmias that interfere with machine triggering/span
Bleeding diathesis
Active thrombophlebitis (Deep vein thrombosis)
Severe lower extremity vaso-occlusive disease
Presence of a documented aortic aneurysm requiring surgical repair
Pregnancy
Severe aortic insufficiency
An abdominal aortic aneurysm greater than 5 cm in diameter
Malignant Hypertension
Hemophilia
Fever
An open leg wound
Superficial phlebitis

### Precautions

Patients with blood pressure higher than 180/110 mmHg should be controlled prior to treatment with enhanced external counterpulsation
Patients with a heart rate more than 120 bpm should be controlled prior to treatment with enhanced external counterpulsation
Patients at high risk of complications from increased venous return should be carefully chosen and monitored during treatment with enhanced external counterpulsation. Decreasing cardiac afterload by optimizing diastolic augmentation may help minimize increased cardiac filling pressures due to venous return.
Patients with clinically significant valvular disease should be carefully chosen and monitored during treatment with enhanced external counterpulsation. Certain valve conditions, such as significant aortic insufficiency, or severe mitral or aortic stenosis, may prevent the patient from obtaining benefit from diastolic augmentation and reduced cardiac afterload in the presence of increased venous return.

Image enhanced external counter pulsation system 10 can monitor the following valvular functions:
Mitral Stenosis
Mitral Regurgitation
Mitral Valve Prolapse
Aortic Stenosis
Aortic Regurgitation
Tricuspid Stenosis
Tricuspid Regurgitation
Tricuspid Valve Prolapse
Pulmonary Regurgitation
Infective Endocarditis
Prosthetic Heart Valves

Abdominal aortic aneurysms, deep vein thrombosis (DVT), lower extremity vaso-occlusive disease, aortic stenosis and aortic insufficiency may also be monitored.

For example, superficial vein thrombophlebitis may occur spontaneously or as a complication of medical or surgical interventions. Sterile thrombophlebitis limited to the superficial veins rarely is life threatening but a thorough diagnostic evaluation is mandatory because many patients with superficial phlebitis also have occult deep vein thrombosis (DVT) which carries very high rates of morbidity and mortality. Phlebitis should be assumed to involve the deep veins until proven otherwise, because superficial vein thrombophlebitis and deep vein thrombophlebitis share the same pathophysiology, pathogenesis, and risk factors.

Image enhanced external counter pulsation system 10 uses an average of 5 cardiac cycles to minimize differences during the breath cycle and diastolic augmentation. Ultrasound may be performed during ECP treatment for systolic/diastolic ratio valve timing by monitoring and evaluating treatment efficacy.

Ultrasound system 22 and transducer 22T may be used to perform at least Doppler analysis, M-mode and or B-mode ultrasound or when indicated strain rate imaging. Any other suitable mode or ultrasound technique may also be used. Image enhanced external counterpulsation system 10 may also include one or more suitable transducers such as abdominal and chest transducers to enable ultrasound monitoring of any suitable patient system during ECP. Images and other ultrasound data from ultrasound system 22 and ECP driver 20 such as ultrasound image 30 of an organ and or other internal elements such as heart 31 may be presented on screen 15S and may be saved in any suitable memory such as memory 16 M and or transmitted to any remote memory for storage and for later review and or analysis.

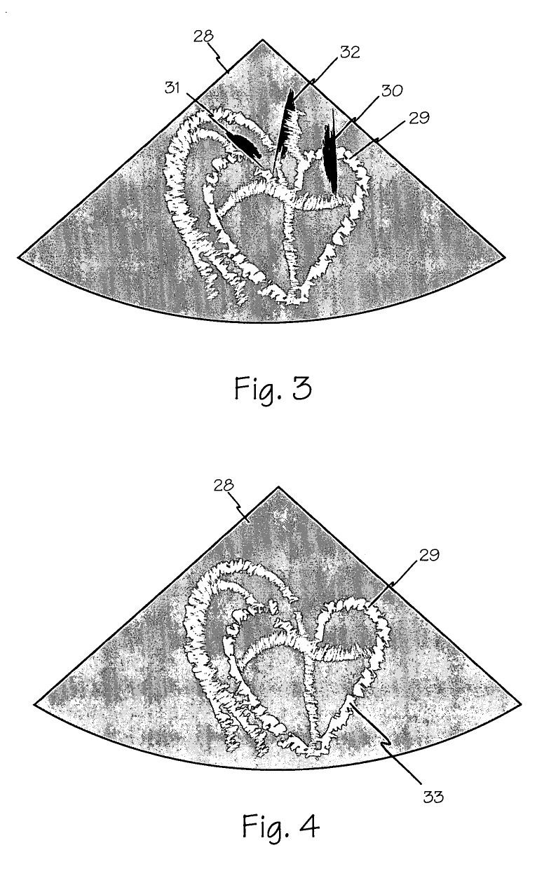

In operation, at the beginning of diastole, inflation cuffs 25, 26 and 27 are sequentially inflated by ECP driver 20 on the calves, thighs and buttocks. This creates retrograde arterial blood flow thru the aorta and into the coronary arteries, improving perfusion to the myocardium. The retrograde blood flow may be visualized for example in FIG. 3 as blood flow 32. There is also an increase venous return which may be visualized for example as blood flow 33 causing an increase in preload. In addition, just prior to systole, cuffs 25, 26 and 27 are quickly deflated, creating a vascular recoil. This causes a vacuum effect, drawing blood down from the aorta which may be visualized as blood flow 34 to the lower extremities dramatically decreasing afterload. The effect is improved cardiac output while reducing workload and oxygen consumption by the myocardium. Thus image enhanced external counterpulsation system 10 presents the ability to capture, save, review and compare ultrasound and tonometry data collected and recorded during ECP therapy.

Referring now to FIG. 4, heart wall sections such as section 35 may be visualized in ultrasound data such as image 30 during ECP therapy. If one or more heart wall sections were hibernating or not contributing to overall heart function ECP therapy may be utilized to identify the hibernating sections such as wall section 35 through ultrasound data collected during ECP therapy. The improved blood perfusion that results from ECP therapy may also improve heart wall function. Storage of one or more images or other ultrasound data such as image 30 in memory 16 M permits an image or other data from a therapy session to be compared to an image or other data from a later therapy session to determine if wall motion or other parameters are improving i.e. cardiac revascularization.

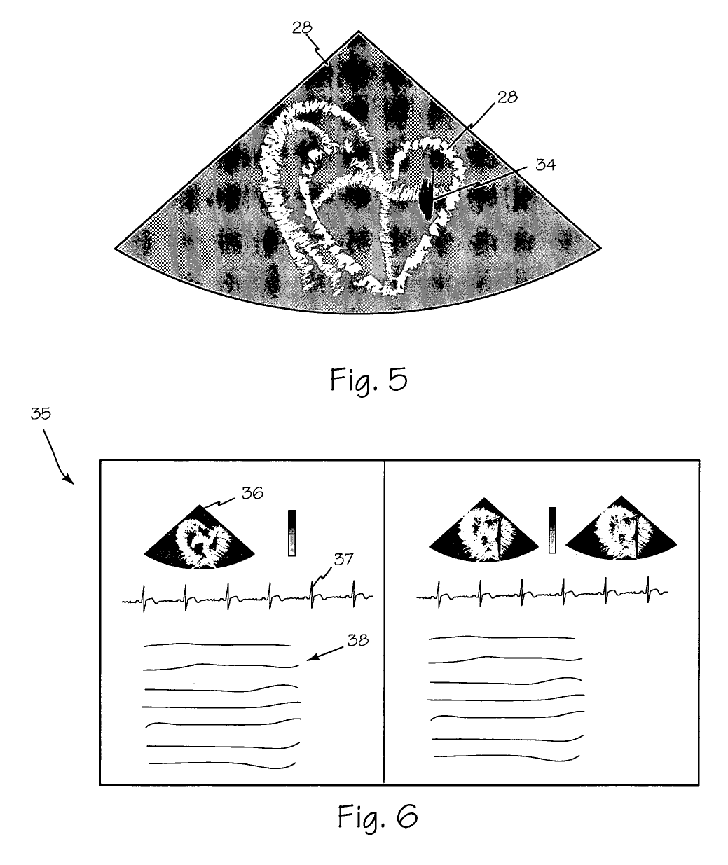

Referring now to FIG. 5, during ECP therapy the timing and intensity of the air flow into cuffs 25, 26 and 27 may be adjusted according to ultrasound data such as image 30. For example, mitral regurgitation evidenced by flow 36 may be considered to adjust the intensity of airflow into the inflation cuffs. Thus patients that may have been poor candidates for the benefits of ECP therapy may be considered for image enhanced external counterpulsation because the therapy may be customized according to the performance of the patient's heart as visualized during the ECP therapy. Other ECP parameters that may be adjusted during therapy informed by ultrasound and or tonometer data may include pressure, cuff placements, timing relative the to the patients heart beats, session duration, preload, afterload and any other suitable parameters.

The combination of ECG, ultrasound, tonometry and ECP to form an image enhanced external counterpulsation system such as system 10 enables the preparation of integrated reports such a report 37 of FIG. 6. Report 35 may include ultrasound data 22D and or tonometry data 23D such as image 38, patient parameters such as ECG 39 and other data 40. Report 37 and its data may be recorded in memory 16 M and or transmitted to one or more remote sites via network link 41 as illustrated in FIG. 1. Thus test data, screen images, and or reports may be viewed remotely in real-time via any suitable LAN or WAN.

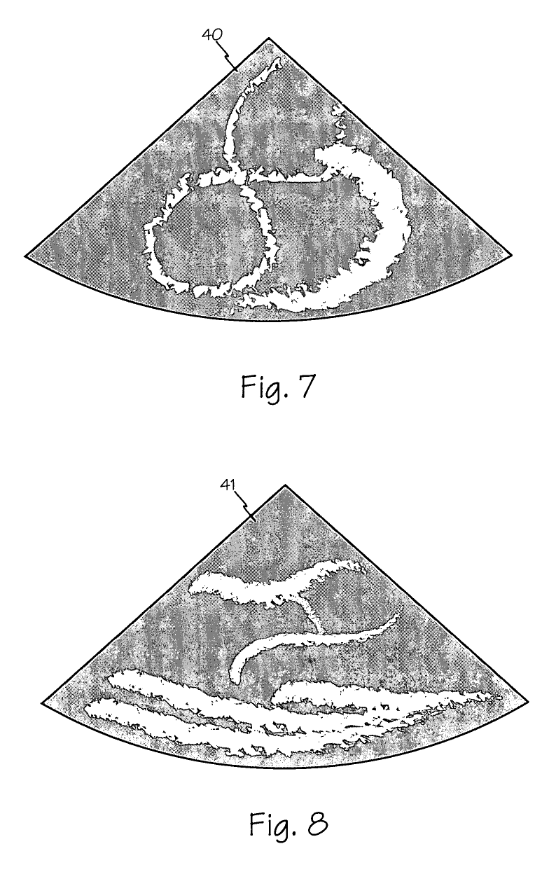

Using an advanced echocardiography technique known as strain rate, wall motion studies can be performed to locate previously hibernating and stunned myocardium. For example, 4-chamber tissue harmonic image 42 of FIG. 7, or long axis tissue harmonic image 43 of FIG. 8 may be used to evaluate strain or strain rate. Systolic deformation (strain) and velocity gradient (strain rate) can be measured to determine if the heart wall is now responding to the improved blood perfusion due to ECP therapy (angiogenesis). Of particular interest in locating previously hibernating myocardium is quantifying myocardial function with an objective assessment of regional function.

Hibernating myocardium is defined as a region of depressed myocardial contractility at rest due to persistently impaired coronary blood flow. It can occur in chronic stable or unstable angina, acute myocardial infarction, heart failure with and without severe LV dysfunction, and an anomalous coronary artery. Therapeutic use of an image enhanced external counterpulsation system such as system 10 may reverse the effects of hibernating myocardium and or stunned myocardium.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A therapeutic and diagnostic device comprising:
an examination table for supporting a patient undergoing ECP therapy;
one or more inflation cuffs for ECP therapy;
an ECP driver with at least one hose for inflating and deflating at least one of the one or more inflation cuffs;
an ultrasound driver and transducer for obtaining ultrasound data from a patient undergoing ECP therapy; and
a control system for simultaneously controlling the ECP driver and the ultrasound driver to obtain ultrasound data of a patient undergoing external counterpulsation therapy.

2. The device of claim 1 further comprising:
an electrode array suitable for collecting ECG data; and
an ECG module for capturing electrocardiograph data from the electrode array.

3. The device of claim 1 further comprising:
a micromanometer for measuring one or more circulatory system parameters; and
a tonometry module for collecting one or more circulatory system parameters.

4. The device of claim 2 further comprising:
a micromanometer for measuring one or more circulatory system parameters; and
a tonometry module for collecting one or more circulatory system parameters.

5. The device of claim 1 wherein the one or more inflation cuffs comprise calf, thigh and buttocks inflation cuffs.

6. The device of claim 1 wherein the ultrasound driver and transducer operate in Doppler mode.

7. The device of claim 1 wherein the ultrasound driver and transducer operate in M-mode.

8. The device of claim 1 wherein the ultrasound driver and transducer operate in B-mode.

9. The device of claim 1 wherein the ultrasound driver and transducer perform strain rate imaging.

10. The device of claim 1 further comprising one or more storage elements for storing ultrasound data.

11. The device of claim 2 further comprising one or more storage elements for storing ultrasound and ECG data.

12. The device of claim 4 further comprising one or more storage elements for storing ultrasound, ECG and tonometry data.

13. A method for optimizing ECP therapy comprising the steps:
securing a patient in one or more inflation cuffs;
connecting an ECP driver to at least one of the one or more inflation cuffs;
operating the ECP driver on a first setting to sequentially inflate and deflate the at least one of the one or more inflation cuffs;
examining the patient using ultrasound during the operation of the ECP driver to collect ultrasound data reflecting the effect of ECP on the patient; and
adjusting the ECP driver according to the ultrasound data.

14. The method of claim 13 further comprising the step of:
storing the ultrasound data for later analysis.

15. A method for performing stress ultrasound comprising the steps:
locating a patient on an ECP therapy table;
securing; a patient in calf inflation cuffs, thigh inflation cuffs and buttocks inflation cuffs;
connecting an ECP driver to calf, thigh and buttocks inflation cuffs using one or more pneumatic hoses;
operating the ECP driver to sequentially inflate and deflate the calf, thigh and buttocks inflation cuffs;
examining the patient using Doppler ultrasound during the operation of the ECP driver to collect ultrasound data;
presenting the ultrasound data on one or more displays; and
storing the ultrasound data in a memory for later review and analysis.
